(19) United States
(12) Patent Application Publication
(10) Pub. No.: US 2016/0003024 A1
(43) Pub. Date: Jan. 7, 2016

(54) ENHANCED OIL RECOVERY FROM A CRUDE HYDROCARBON RESERVOIR

(71) Applicant: HALDOR TOPSØE A/S, Kgs. Lyngby (DK)

(72) Inventors: Henrik Udesen, Holte (DK); Angelica Hidalgo Vivas, Herlev (DK)

(73) Assignee: Haldor Topsøe A/S, Kgs. Lyngby (DK)

(21) Appl. No.: 14/652,292

(22) PCT Filed: Feb. 13, 2013
(86) PCT No.: PCT/EP2013/052840

§ 371 Date: Jun. 15, 2015

(51) Int. Cl. E21B 43/40; C10G 3/00; E21B 43/16; C07C 29/151; C07C 41/09; C10G 2/00; C07C 41/01

(52) U.S. Cl. CPC E21B 43/40; C10G 2/30; C10G 3/49; C07C 41/01; C07C 29/151; C07C 41/09; E21B 43/16

(57) ABSTRACT

The invention relates to a method and a system for recovery of oil from a crude hydrocarbon reservoir. A synthesis gas from natural gas, and then liquid hydrocarbon or liquid oxygenate is produced from said synthesis gas. The liquid hydrocarbon or liquid oxygenate is then passed into said crude hydrocarbon reservoir to provide a crude hydrocarbon mixture, and the crude hydrocarbon mixture is withdrawn from said reservoir.

### FIELD OF THE INVENTION

The invention relates to a method and a system for recovery of oil from a crude hydrocarbon reservoir.

### BACKGROUND TO THE INVENTION

Natural gas is a naturally-occurring hydrocarbon gas mixture consisting primarily of methane, together with other hydrocarbons, carbon dioxide, nitrogen and hydrogen sulfide.

Crude hydrocarbon reservoirs usually comprise a mixture of hydrocarbon liquids (i.e. crude oil, including dissolved gases) and natural gas. Unwanted natural gas often comprises a disposal problem in oil fields, as it has to be purified and transported before it can be put to commercial use. For instance, non-hydrocarbons such as carbon dioxide, nitrogen, helium (rarely), and hydrogen sulfide must also be removed before the natural gas can be transported.

Often, purification and transport of natural gas is simply not commercially viable,—especially at remote oil fields—and it is instead burnt off at the oil field. However, additional increasing environmental regulation can limit the burning of natural gas. As an alternative, the natural gas can be pumped back into the underground reservoir in order to preserve pressure of the reservoir. By preserving the reservoir pressure the recoverable fraction normally increases.

It would be of interest if the natural gas obtained as a by-product of oil extraction could be put to good use instead of being burnt off or otherwise disposed of. It would be advantageous if the natural gas could be used on-site at the oil field and more advantageous if it could be used in connection with a process that increases the recoverable fraction of the crude hydrocarbon.

### SUMMARY OF THE INVENTION

The present invention thus provides a method for oil recovery from a crude hydrocarbon reservoir, said method comprising the steps of:
a. providing a natural gas,
b. producing a synthesis gas from said natural gas,
c. producing liquid hydrocarbons or liquid oxygenates from said synthesis gas,
d. passing said liquid hydrocarbons or liquid oxygenates into said crude hydrocarbon reservoir to provide a crude hydrocarbon mixture, and
e. extracting said crude hydrocarbon mixture from said crude hydrocarbon reservoir.

The invention also provides a system comprising a gas-to-liquids (GTL) plant connected to a crude hydrocarbon reservoir, said GTL plant comprising:
a. a process unit for producing synthesis gas from natural gas,
b. a synthesis unit, e.g., a TIGAS® unit, connected to said process unit, said synthesis unit arranged for producing liquid hydrocarbon or liquid oxygenate from said synthesis gas,
said system comprising:
c. means for connecting the crude hydrocarbon reservoir with the process unit and arranged to transport natural gas from said reservoir to said process unit, and
d. means for connecting the synthesis unit with said crude hydrocarbon reservoir and arranged to pass liquid hydrocarbon or liquid oxygenate from synthesis unit into said crude hydrocarbon reservoir.

Further details of the method and system of the invention can be found in the following description of the invention, the figures and the dependent claims.

### FIGURES

FIG. 1 is a schematic illustration of one embodiment of the system of the invention.

FIG. 2 is a schematic illustration of another embodiment of the system of the invention.

FIG. 3 shows the TIGAS® gasoline composition used in Example 2.

FIG. 4 charts the viscosity of an HVGO mixture following dilution as per Example 2.

### DETAILED DESCRIPTION OF THE INVENTION

As set out above, the invention provides a method for oil recovery from a crude hydrocarbon reservoir. The crude hydrocarbon reservoir is underground, and may also be subocean.

The first step of the method is the provision of natural gas. Ideally, the natural gas is obtained from the crude hydrocarbon reservoir. However, it may also be possible that an external supply of natural gas is provided. External supplies of natural gas may be provided from a nearby natural gas or crude oil reservoir.

The present invention makes use of gas-to-liquid (GTL) technology, in which gaseous hydrocarbons are converted into liquid hydrocarbons or liquid oxygenates. The “gaseous” and “liquid” states are measured at normal temperature and pressure (NTL) conditions.

In the first step of a GTL process, natural gas is converted to synthesis gas. This takes place via steam methane reforming or partial oxidation of the methane present in the natural gas to synthesis gas. Synthesis gas or “syngas” gas is a gas mixture comprising CO, H₂ and possibly some CO₂ the carbon monoxide (CO) to hydrogen (H₂) ratio in the syngas may be adjusted as required (e.g. using the water gas shift reaction). For liquid hydrocarbon production, the mole ratio of H₂/CO is preferably above 1.

Suitable apparatus for the provision of synthesis gas from natural gas is known to the skilled person, and may—for instance—include one or more auto thermal reformers, pre-reformers, tubular reformers, etc.

The second step of the GTL process is the formation of liquid hydrocarbons or liquid oxygenates from the syngas.

Liquid hydrocarbons may be formed directly from syngas, e.g. in a Fischer-Tropsch process.

Alternatively, liquid hydrocarbons may be formed indirectly from the syngas, via oxygenates. A preferred method for this process is the so-called “Topsoe integrated gasoline synthesis (TIGAS®)” process which converts synthesis gas to gasoline via methanol (MeOH) or MeOH and dimethylether. The TIGAS® technology is described in inter alia U.S. Pat. No. 4,481,305, US2012078023, WO10149339, U.S. Pat. No. 8,067,474, U.S. Pat. No. 8,202,413 and US2010036186. Another MeOH-to-gasoline process is described in U.S. Pat. No. 4,011,275 and U.S. Pat. No. 4,138,442.

To form liquid hydrocarbons in the TIGAS® process, synthesis gas is first converted to either methanol, which is then dehydrated to dimethyl ether (DME), or to a combined MeOH/DME product. Further conversion of said methanol or MeOH/DME produces liquid hydrocarbons, preferably in the presence of a zeolite-type catalyst. The liquid hydrocarbons thus produced may be used directly in the next step of the method (reinjection to the oil well); alternatively, they may be further processed as desired to obtain the liquid hydrocarbon stream to be reinjected to the oil well.

If the product of the GTL process is a liquid hydrocarbon, said liquid hydrocarbons are preferably in the gasoline range, e.g. compounds containing 4-16 carbons, such as 5-12 carbons.

Oxygenates are fuels containing compounds with oxygen in their chemical structures. Typical oxygenates are alcohols and ethers. Alcohols produced in a TIGAS® process may be methanol, ethanol, or mixtures thereof. Ethers produced may be dimethyl ether (DME).

After production of liquid hydrocarbons or liquid oxygenates in the GTL process, the liquid hydrocarbon or liquid oxygenate is passed into the crude hydrocarbon reservoir. Typically, the liquid hydrocarbon or liquid oxygenate is pumped into the reservoir at high pressure, which depends on the depth of the geologic formation, e.g. 100-1400 bar. It therefore mixes with the crude hydrocarbon in the reservoir to provide a crude hydrocarbon mixture.

Liquid hydrocarbons and liquid oxygenates have been proven to work as solvents, whereby more of the heavy fraction of the crude hydrocarbon can be recovered. Improved recovery is achieved by dissolution of heavier fractions. In addition, there is no influence of particulate matter in the crude hydrocarbon mixture (see Examples).

The crude hydrocarbon mixture is then extracted from the reservoir.

It is known to pump liquids such as water into a crude hydrocarbon reservoir to improve crude hydrocarbon recovery. However, such methods require a ready source of water, which then needs to be separated from the crude hydrocarbon in a phase separation stage. Among the many advantages of the present invention is the fact that the liquid hydrocarbon or liquid oxygenate is manufactured in-situ from a by-product of the crude hydrocarbon reservoir, and its hydrocarbon nature means that it can be readily co-processed with the crude hydrocarbons in a refining stage. Indeed, the liquid hydrocarbons or liquid oxygenates contribute to overall production from a hydrocarbon reservoir. In addition, the transportation of the crude hydrocarbon to the refinery is facilitated by the reduction in viscosity achieved by having added the liquid hydrocarbons or liquid oxygenates.

In addition, the amount or chemical composition of the liquid hydrocarbon or liquid oxygenate can be tailored so that the properties (e.g. viscosity, chemical composition) of the mixture is optimised (see Examples). For instance, the lower the C5 content in the liquid hydrocarbon, the lower the risk of precipitation of e.g. asphaltenes.

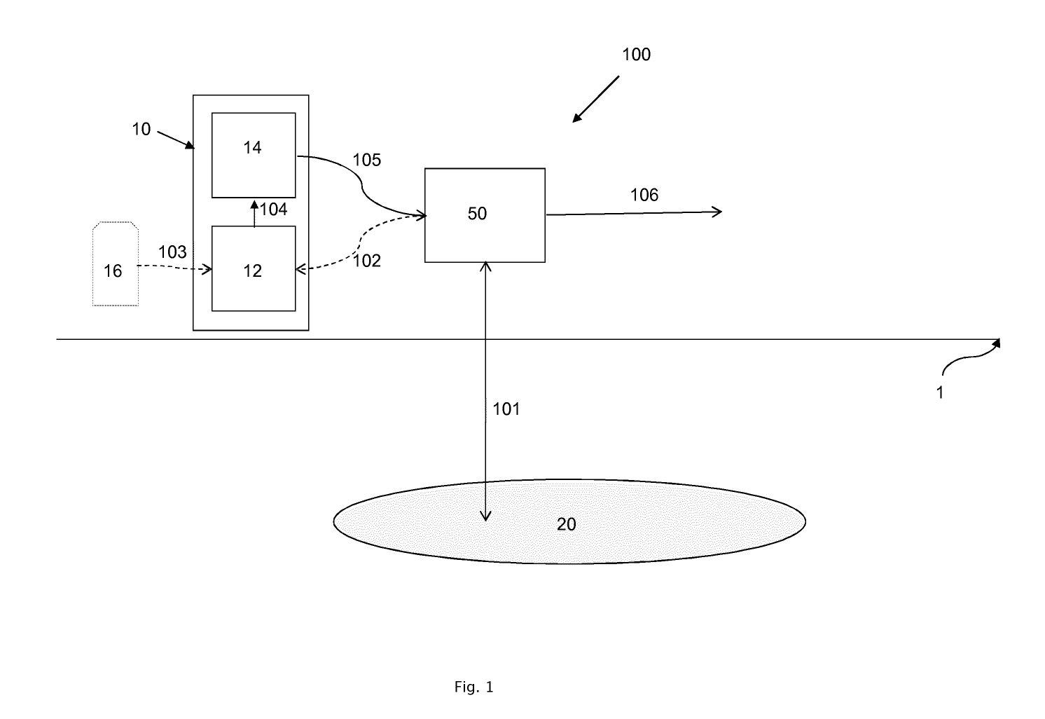

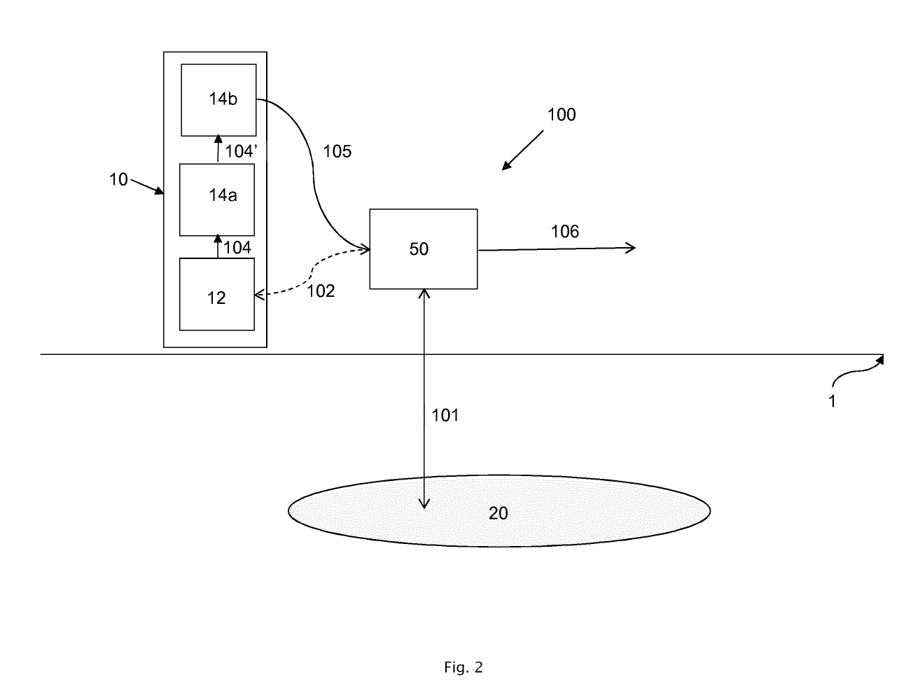

The present invention also provides a system 100 for oil recovery from a crude hydrocarbon reservoir. FIGS. 1 and 2 illustrate the system 100 of the invention in a schematic manner.

The system 100 illustrated in the figures comprises a gas-to-liquids (GTL) plant 10 connected to a crude hydrocarbon reservoir 20. The reservoir 20 is typically underground (illustrated by 1). Connection between the GTL plant 10 and the crude hydrocarbon reservoir is by means of a 2-way conduit for gas and liquids (indicated by reference 101 in FIGS. 1 and 2).

In the embodiment shown in FIG. 1, the GTL plant 10 comprises:
a. a process unit 12 arranged for producing synthesis gas from natural gas,
b. a synthesis unit 14 connected to said process unit, said synthesis unit 14 arranged for producing liquid hydrocarbons or liquid oxygenates from said synthesis gas.

The process unit 12 is configured with flow means 102, 103, whereby it can receive natural gas. Preferably, the natural gas fed into said process unit 12 is obtained from said hydrocarbon reservoir 20 (and fed via flow means 102). Optionally, an additional natural gas source 16 may be used to provide all or part of the natural gas fed into said process unit 12 (via flow means 102). Process unit 12 typically takes the form of one or more reformer units, e.g. autothermal reformers, pre-reformers, tubular reformers, convection reformers, etc.

From the process unit 12, synthesis gas is passed to a synthesis unit 14. The synthesis unit 14 is configured with flow means 104, whereby it can receive synthesis gas from said process unit 12. The synthesis unit 14 is therefore connected to said process unit 12, and arranged to produce liquid hydrocarbons or liquid oxygenates from said synthesis gas.

The synthesis unit (14) is suitably a Fischer-Tropsch (F-T) unit, a TIGAS®-methanol to gasoline (MTG) unit or a TIGAS®-synthesis gas to gasoline (STG) unit

The system 100 is also configured with flow means 105, 101 such that said liquid hydrocarbon or liquid oxygenate can be passed from said synthesis unit 14 to said crude hydrocarbon reservoir 20.

In the crude hydrocarbon reservoir 20, the liquid hydrocarbon or liquid oxygenate forms a mixture with the crude hydrocarbon. This mixture can then be withdrawn (i.e. pumped) from the reservoir 20. Extraction of the crude hydrocarbon mixture from the crude hydrocarbon reservoir is typically carried out via flow means 101, and the mixture is sent for further processing (e.g. refining) via flow means 106.

As set out above, the organic nature of the liquid hydrocarbon or liquid oxygenate means that it can be readily separated from the crude hydrocarbon in a refining stage. The system of the invention may additionally comprise refining means for refining the crude hydrocarbon mixture extracted from the crude hydrocarbon reservoir.

Typically, flow means 101, 102, 103, 104, 105, 106 take the form of one or more pipes or conduits, together with storage tanks, valves, pumps and other elements as required.

As shown in FIGS. 1 and 2, the system 100 according to the invention comprises a pump unit 50 located between the (GTL) plant 10 and the crude hydrocarbon reservoir 20. The pump unit 50 allows fluids (i.e. liquids and gases) to be pumped to and from the crude hydrocarbon reservoir 20 via flow means 101. The pump unit 50 also allows fluids to be pumped to the process unit 12, and from the synthesis unit 14. Control means within pump unit 50 allow the appropriate flow of fluid to be selected and controlled.

Natural gas is often obtained from the crude hydrocarbon reservoir in a mixture or dissolved in crude hydrocarbon and/or water. In such instances, it is therefore desirable to separate the natural gas from other components, prior to processing in the process unit 12. In one embodiment, therefore, the pump unit 50 also comprises a separation unit arranged so as to separate natural gas from the crude hydrocarbon prior to supplying it to the process unit 12.

In FIG. 2, the synthesis unit 14 comprises oxygenate synthesis unit 14a and gasoline synthesis unit 14b. The oxygenate synthesis unit 14a is configured with flow means 104 whereby it can receive synthesis gas the said process unit 12. The oxygenate synthesis unit 14a being arranged for producing liquid oxygenates from the synthesis gas.

Gasoline synthesis unit 14b is configured with flow means 104′, whereby it can receive said oxygenates from the oxygenate synthesis unit 14a. The gasoline synthesis unit 14b is arranged for producing liquid gasoline from said oxygenates.

Synthesis gas therefore passes from the process unit 12 to the oxygenate synthesis unit 14a, in which it is converted into liquid oxygenates. The liquid oxygenates from the oxygenate synthesis unit 14a are passed to the gasoline synthesis unit 14b, in which they are then converted into liquid hydrocarbons.

Similar to the embodiment of FIG. 1, the gasoline synthesis unit 14b is configured with flow means 105, 101 such that said liquid gasoline can be passed from said gasoline synthesis unit 14b to said crude hydrocarbon reservoir 20.

Suitable components for the synthesis units 14a, 14b are described in the above-mentioned documents relating to TIGAS®.

Due to the compact, self-contained nature of the system of the invention, it can be readily incorporated into existing plants, rigs and platforms for crude hydrocarbon recovery. The present invention thus relates to an oil platform or a floating production, storage off-loading facility (FPSO) comprising the system according to the invention.

All features of the method of the invention are also relevant for the system of the invention.

The method and system of the invention will in addition also enhance the transportation of the crude hydrocarbons, e.g., to a refinery. The advantages include:
Significant viscosity reduction at relatively low dilution ratios, thus pipe diameter is not significantly increased compared to undiluted crude hydrocarbon
Less power consumption when pumping/transporting the crude hydrocarbon mixture in the pipe due to lower viscosity
Corrosion in the wellhead (during extraction) or pipeline (during) can be reduced, as water is not pumped into the reservoir or pipeline.
An investment adds value, as the diluent (oxygenates or liquid hydrocarbons) can be sold as a commercial product after recovery in a refinery
“Flaring” of natural gas is avoided, giving environmental benefits.

### EXAMPLES

### Example 1

A sample of bitumen of Canadian origin with a content of fines of 0.7 wt. % and high viscosity at room temperature (viscosity @ 100° F.>1230 cSt) was diluted with a model TIGAS® gasoline with the composition shown in Table 1.

TABLE 1
Synthetic TIGAS ® gasoline.
TIGAS ® model gasoline for diluent studies

| Compound | wt % |
|---|---|
| 2-methylbutane | 17.9 |
| n-pentane | 2.0 |
| Hexane, isomer mixture | 22.9 |
| Methycyclopentane | 1.0 |
| Benzene | 0.1 |
| n-heptane | 16.8 |
| Methylcyclohexane | 2.1 |
| Toluene | 1.0 |
| n-octane | 2.0 |
| i-octane | 4.0 |
| Ethyl-Cy-C6 | 2.0 |
| Ethylbenzene | 0.8 |
| o,m,p-xylene | 9.1 |
| i-propyl-Cy-C6 | 2.0 |
| 124-TMBz | 7.4 |
| Durene | 8.6 |
| PMB | 12.0 |
| | 100.0 |

Dilution experiments were carried out by mixing known amounts of diluent and bitumen for several hours.

The viscosity of two mixtures of diluted bitumen of 30 wt % and 50 wt %, respectively, were determined with reproducible results, indicating that there was no influence of particulate matter in the samples, i.e., no asphaltenes precipitation. Particles in samples will normally lead to large standard deviations, and viscosity measurements have been used in the determination of particle flocculation in crude hydrocarbons. Viscosities are measured according to ASTM D 7042 and are given in Table 2. The viscosity of the neat bitumen at room temperature is very high (>1230 cSt), and the results thus indicate that addition of 30% diluent results in a significant reduction in viscosity.

TABLE 2
Viscosity of TIGAS ® diluent/bitumen mixtures.

| | Viscosity cSt |
|---|---|
| Dilution wt % | at 20° C. |
| 30 | 43.2 |
| 50 | 6.2 |

As can be seen, even at relatively low dilutions (of ca. 30 wt %), low viscosity mixtures can be obtained.

### Example 2

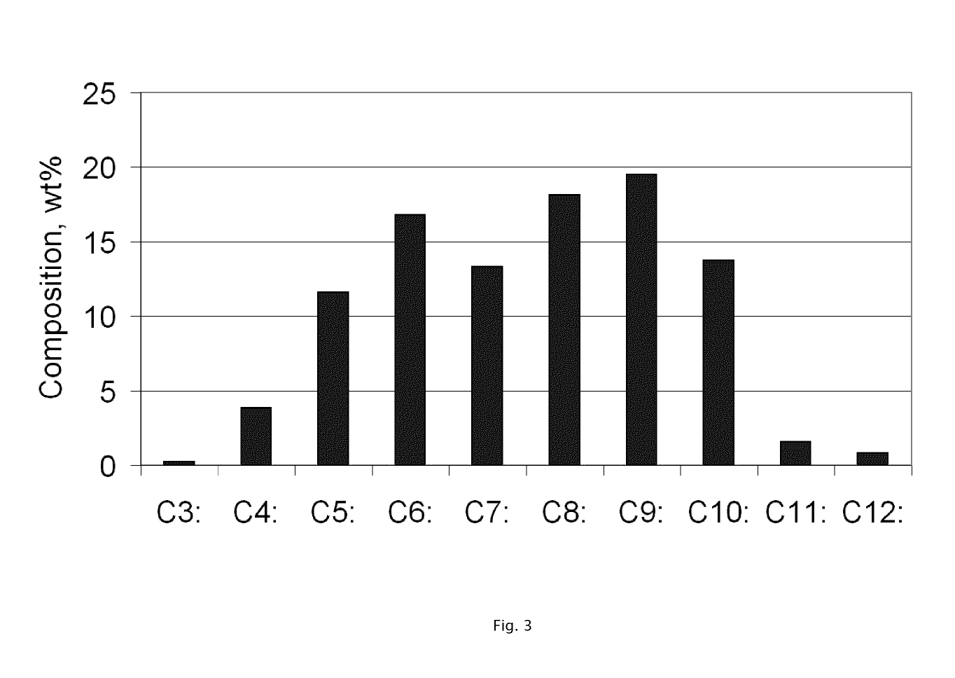

A heavy vacuum gas oil fraction with a viscosity of 460 cSt at 40° C. was diluted with a TIGAS® gasoline with the composition shown in FIG. 3.

Dilution experiments were carried out by mixing known amounts of diluent and heavy vacuum gas oil (HVGO) and measuring viscosity at 40° C. according to method ASTM D 7042.

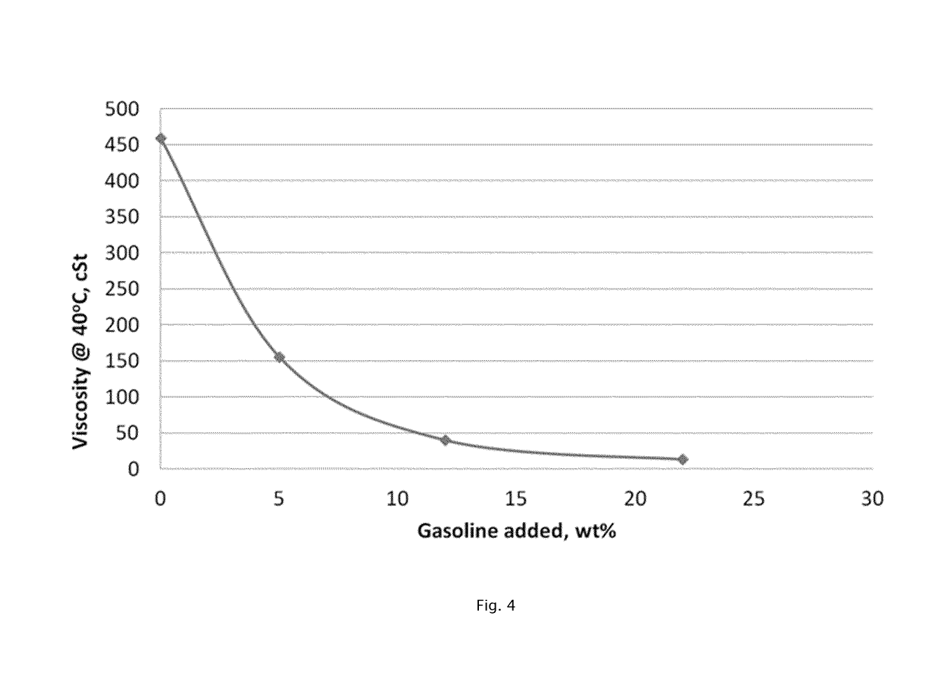

Viscosity is significantly reduced, even when only a small amount of gasoline (ca. 5 wt %) is added (see FIG. 4).

| Gasoline added, | Viscosity at |
|---|---|
| wt % | 40° C., cSt |
| 0 | 458.57 |
| 5 | 155.24 |
| 12 | 40.058 |
| 22 | 13.36 |

1. A method for oil recovery from a crude hydrocarbon reservoir, said method comprising the steps of:
a. providing a natural gas,
b. producing a synthesis gas from said natural gas,
c. producing liquid hydrocarbons or liquid oxygenates from said synthesis gas,
d. passing said liquid hydrocarbons or liquid oxygenates into said crude hydrocarbon reservoir to provide a crude hydrocarbon mixture, and
e. extracting said crude hydrocarbon mixture from said crude hydrocarbon reservoir.

2. The method according to claim 1, wherein step c. comprises the steps of:
c1. converting synthesis gas to methanol or methanol/dimethylether (DME),
c2. dehydrating said methanol or MeOH/DME to dimethyl ether
c3. further dehydrating said dimethyl ether to form liquid hydrocarbons, preferably in the presence of a zeolite catalyst.

3. The method according to claim 1, wherein the natural gas in step a. is itself obtained from said crude hydrocarbon reservoir.

4. The method according to claim 3, wherein the natural gas is separated from the crude hydrocarbons prior to being used in step a.

5. The method according to claim 1, wherein liquid hydrocarbons are produced from said synthesis gas in step c.

6. The method according to claim 5, wherein said liquid hydrocarbons are in the gasoline range, i.e. C5-C12,

7. The method according to claim 2, wherein liquid oxygenates (MeOH or MeOH/DME) are produced from said synthesis gas in step c.

8. The method according to claim 7, wherein said liquid oxygenate is methanol, ethanol, DME or a mixture thereof.

9. A system comprising a gas-to-liquids (GTL) plant connected to a crude hydrocarbon reservoir, said GTL plant comprising:
a. a process unit arranged for producing synthesis gas from natural gas,
b. a synthesis unit connected to said process unit, said synthesis unit arranged for producing liquid hydrocarbons or liquid oxygenates from said synthesis gas,
wherein the process unit is configured with flow means whereby it can receive natural gas; and
wherein the synthesis unit is configured with flow means whereby it can receive synthesis gas from said process unit; and
wherein the system is also configured with flow means such that said liquid hydrocarbons or liquid oxygenates can be passed from said synthesis unit to said crude hydrocarbon reservoir.

10. The system according to claim 9, wherein a pump unit is located between the GTL plant and the crude hydrocarbon reservoir.

11. The system according to claim 10, wherein said pump unit also comprises a separation unit arranged so as to separate natural gas from the crude hydrocarbon prior to supplying it to the process unit.

12. The system according to claim 9, wherein the natural gas fed into said process unit is obtained from said hydrocarbon reservoir.

13. The system according to claim 9, wherein the synthesis unit comprises:
a. oxygenate synthesis unit, wherein said oxygenate synthesis unit is configured with flow means whereby it can receive synthesis gas from said process unit, said oxygenate synthesis unit being arranged for producing liquid oxygenates from said synthesis gas; and
b. gasoline synthesis unit, wherein said gasoline synthesis unit is configured with flow means whereby it can receive said liquid oxygenates from said oxygenate synthesis unit, said gasoline synthesis unit being arranged for producing liquid gasoline from said liquid oxygenates;
wherein the gasoline synthesis unit is also configured with flow means such that said liquid gasoline can be passed from said gasoline synthesis unit to said crude hydrocarbon reservoir.

14. The system according to claim 9, wherein the synthesis unit is a Fischer-Tropsch unit, a TIGAS®-MTG unit or a TIGAS®-STG unit.

15. An oil platform or FPSO comprising the system according to claim 9.
